(19) United States
(12) Patent Application Publication
(10) Pub. No.: US 2023/0241357 A1
(43) Pub. Date: Aug. 3, 2023

(54) GUIDE WIRE SHAPING TOOL AND GUIDE WIRE SHAPING METHOD

(71) Applicants: ST. MARIANNA UNIVERSITY SCHOOL OF MEDICINE, Kawasaki-shi (JP); OZK CO., LTD., Yao-shi (JP); SHINWA SYOJI CO., LTD., Higashiosaka-shi (JP)

(72) Inventors: Yuki ISHIBASHI, Kawasaki-shi (JP); Yoshihiro AKASHI, Kawasaki-shi (JP); Haruhiko YAMASAKI, Yao-shi (JP); Katsunori MITSUHASHI, Yao-shi (JP); Yoshiro MORISHITA, Higashiosaka-shi (JP)

(21) Appl. No.: 18/155,727

(22) Filed: Jan. 17, 2023

(30) Foreign Application Priority Data
Jan. 21, 2022 (JP) 2022-008026

(51) Int. Cl. A61M 25/09; A61L 31/14

(52) U.S. Cl. CPC A61M 25/09041; A61L 31/14; A61M 2025/09108

(57) ABSTRACT

An inlet passage portion, which is a substantially columnar space through which a tip end of a guide wire is inserted into an opening portion to pass the guide wire, a shaping portion, which is a space communicating with the inlet passage portion through an outlet portion, which is a portion located at a deepest part of the inlet passage portion, and flatly expanding from the outlet portion, an annular inner wall, which forms an inner circumferential portion of the shaping portion, an extension line intersection portion, which is an inner wall intersecting an extension line of a center line of the inlet passage portion at an obtuse angle, and an obtuse angle side inner wall portion, which is an inner wall extending from the outlet portion to the extension line intersection portion and forming an obtuse angle with the extension line are included.

### BACKGROUND OF THE INVENTION

### Field of the Invention

The present invention relates to a guide wire shaping tool and a guide wire shaping method, and more particularly to a guide wire shaping tool that performs shaping by inserting a guide wire into a shaping portion that is a flatly expanding space, and a guide wire shaping method using this guide wire shaping tool.

### Description of Related Art

Conventionally, there is a guide wire shaping tool configured such that a tip end of a guide wire is inserted into an opening portion of an inlet passage portion, and the tip end protruding from this inlet passage portion is pressed against a pressing die to form a desired shape (JP 2014-68965 A).

The guide wire refers to a flexible, wire-like instrument for facilitating insertion and indwelling of a catheter introducer into a blood vessel. Then, by the user performing shaping process so as to fold back the tip end of the guide wire using the guide wire shaping tool, the risk that the guide wire damages an inner wall of the blood vessel of the patient is reduced. Since a heating treatment process, a chemical treatment process, and the like are unnecessary in order to perform mechanical shaping by the guide wire shaping tool as described above, the user can perform a shaping process also in a medical site such as an operating room, for example, and does not need to select a work place.

The inventor of the present invention is the same inventor as the inventor of the invention according to International Application No. PCT/JP2020/027748, the international publication date of which is Jan. 28, 2021. The patent application to which this description is attached is made within one year from the International Publication Date.

### SUMMARY OF THE INVENTION

Some guide wires include a flat-plate-like core wire in order to ensure the fastness. FIGS. 1 is an example of a guide wire having such a flat-plate-like core wire, where FIG. 1A shows an example of a guide wire in which a resin plastic jacket covers around a metal core wire, and FIG. 1B shows an example of a guide wire in which a coil is wound around the core wire. Examples of the material of the metal core wire include stainless steel, and examples of the material of the plastic jacket include polytetrafluoroethylene.

In the case of a guide wire having such a flat-plate-like core wire, the ease of bending of the guide wire differs depending on the relationship between the direction in which the guide wire is bent and the long direction or the short direction in the transverse cross section of the core wire. Specifically, when the short direction of the core wire in the transverse cross section coincides with the up-down direction as in the upper view of FIG. 2A, that is, when the long direction and the bending direction of the guide wire are the same as in the lower view of FIG. 2A, the guide wire is hardly bent left and right, and a large force needs to be applied in order to forcibly bend the guide wire. Conversely, when the long direction of the core wire in the transverse cross section coincides with the up-down direction as in the upper view of FIG. 2B, that is, when the short direction and the bending direction of the guide wire are the same as in the lower view of FIG. 2B, the guide wire is easily bent left and right as compared with the case of FIG. 2A.

On the other hand, for example, the plastic jacket is often colored, and it is difficult to determine the long direction or the short direction in the transverse cross section of the core wire only by viewing the guide wire from the outside. Therefore, when the guide wire is inserted into the guide wire shaping tool and the tip end is to be shaped, it is easy to perform shaping when the core wire is arranged in a direction in which the guide wire is easily bent as in FIG. 2B, but when the core wire is arranged in a direction in which the guide wire is difficult to be bent as in FIG. 2A, efficiency of the shaping is poor, and there is a possibility that the guide wire cannot be shaped into a desired shape. In this manner, a situation occurs in which there is a variation between the difficulty of the shaping process of the guide wire and the quality of the shaping depending on the direction of the core wire.

In view of such a problem, an object of the present invention is to provide a guide wire shaping tool and a guide wire shaping method that can obtain uniform difficulty in shaping process of a guide wire and quality of shaping regardless of the direction of a core wire inside a guide wire to be inserted into the guide wire shaping tool when performing shaping of folding back a tip end of the guide wire.

The present invention is to provide a guide wire shaping tool including: an inlet passage portion that is a substantially columnar space through which a tip end of a guide wire is inserted into an opening portion to allow the guide wire to pass; a shaping portion that is a space communicating with the inlet passage portion through an outlet portion that is a portion located at a deepest part of the inlet passage portion and flatly expanding from the outlet portion; an annular inner wall forming an inner circumferential portion of the shaping portion; an extension line intersection portion that is, of the inner wall, an inner wall intersecting with an extension line of a center line of the inlet passage portion at an obtuse angle; an obtuse angle side inner wall portion that is, of the inner wall, an inner wall extending from the outlet portion to the extension line intersection portion and is an inner wall at an obtuse angle with the extension line; and an acute angle side inner wall portion that is, of the inner wall, an inner wall extending from the outlet portion to the extension line intersection portion and is an inner wall at an acute angle with the extension line, in which the guide wire is configured such that, as the guide wire is fed from the opening portion, the tip end that is sequentially inserted into the inlet passage portion and the shaping portion abuts against a vicinity of the extension line intersection portion, and at a time of this abutment, the guide wire slides along the obtuse angle side inner wall portion while being bent on the obtuse angle side, and an entire guide wire is annularly arranged.

As a result of intensive studies, the present inventor has found that when the tip end of the inserted guide wire abuts on the vicinity of the extension line intersection portion and slides along the inner wall while being bent on the obtuse angle side, even if the long direction of the transverse cross section of the flat-plate-like core wire inside the guide wire is substantially the same as the bending direction of the guide wire and the core wire is located at a position as in FIG. 2A, the guide wire rotates by 90° about its center axis, and has completed the present invention. As a result of the rotation, the short direction of the cross section of the core wire and the bending direction of the guide wire become the same, and therefore the guide wire is easily bent as compared with that before the rotation. It has been found that even when the long direction of the cross section of the core wire and the bending direction of the guide wire form an angle of equal to or less than 90° even not the same, the guide wire rotates by the angle of equal to or less than 90°. As a result of the rotation, similarly, the short direction of the cross section of the core wire and the bending direction of the guide wire become the same, and therefore the guide wire is easily bent as compared with that before the rotation.

In this manner, regardless of the direction of the core wire inside the guide wire when inserted into the guide wire shaping tool of the present invention, the guide wire abuts on the vicinity of the extension line intersection portion and slides along the inner wall, whereby the guide wire can change its attitude so as to be easily bent. Therefore, the guide wire shaping tool of the present invention can achieve uniform ease of shaping process of a guide wire and quality of shaping.

The acute angle side inner wall portion may have a shape bulging in an orientation away from the extension line.

That is, since the inner wall of the guide wire shaping tool of the present invention has the acute angle side inner wall portion bulging in an orientation away from the extension line, when the tip end of the guide wire slides along the inner wall while being bent on the obtuse angle side, a portion continuous with the tip end of the guide wire can be curved so as to approach or contact the acute angle side inner wall portion. Due to this, as compared with a case where the continuous part is not curved, the tip end can smoothly slide while making a shallower angle with respect to a wall surface direction of the inner wall. Therefore, the guide wire can more easily rotate and can be more efficiently shaped.

In this manner, the guide wire shaping tool of the present invention can achieve uniform ease of shaping process of a guide wire and quality of shaping.

An obtuse angle side outlet portion that is one side portion on a side having a substantially linear inner wall continuous with the obtuse angle side inner wall portion, of the outlet portion having a width that allows the guide wire to be doubly arranged; an acute angle side outlet portion that is one side portion on a side opposite to the obtuse angle side outlet portion, of the outlet portion; and a holding portion configured to press and hold the tip end and its vicinity against a main body of the guide wire shaping tool at the obtuse angle side outlet portion may be included, and the guide wire may be partially pulled out from the opening portion, remaining in a state where the tip end and its vicinity are held by the holding portion, and a part of the guide wire annularly arranged inside the shaping portion may be discharged through the acute angle side outlet portion and a remaining portion may be annularly shaped with a smaller diameter.

The user of the guide wire shaping tool of the present invention feeds the guide wire, and after the tip end of the guide wire slides along the obtuse angle side inner wall portion and reaches the outlet portion, conversely, the user partially pulls out the guide wire from the opening portion. In this manner, the guide wire can be annularly shaped by reducing the annular diameter of the guide wire inside the shaping portion. At this time, the position of the tip end can be reliably fixed by the holding portion holding the tip end of the guide wire, and the tip end is held at the obtuse angle side outlet portion of one side portion, whereby the guide wire to be pulled out can be passed through the acute angle side outlet portion of the other side portion. Therefore, it becomes possible to perform shaping by reducing the annular diameter of the guide wire more efficiently.

An obtuse angle side outlet opposing portion and an acute angle side outlet opposing portion that are inner walls opposing each other in the obtuse angle side outlet portion and the acute angle side outlet portion; and a sliding portion in which a part including the acute angle side outlet opposing portion and an acute angle side outlet adjacent portion that is a part adjacent to the acute angle side outlet opposing portion of the acute angle side inner wall portion is nested, the sliding portion being slidable along a groove-shaped rail portion provided in a direction orthogonal to the center line may be included, and the sliding portion may slide, and an interval between the obtuse angle side outlet opposing portion and the acute angle side outlet opposing portion and an interval between the obtuse angle side inner wall portion and the acute angle side outlet adjacent portion may increase.

When the guide wire is inserted into a blood vessel, the load on the blood vessel is reduced by folding back the tip end in a J shape. However, when shaping of folding back the guide wire into the J shape is performed, if the force applied to bend the guide wire at the time of folding back is too large, stress in a specific orientation remaining after plastic deformation is large. Due to this, there is a case where after the guide wire is removed from the guide wire shaping tool, the folded back part reaches a state of being bent to hang down the neck. That is, a state occurs in which the guide wire in which the tip end is folded back and shaped along the obtuse angle side inner wall portion while being bent on the obtuse angle side is further curved in the orientation of folding back together with the folded back part. A state in which stress in a specific orientation due to such plastic deformation excessively remains is also referred to as state in which “ironing” is applied.

In particular, when shaping is performed with reducing the annular diameter of the guide wire, if the interval between the obtuse angle side outlet opposing portion and the acute angle side outlet opposing portion and the interval between the obtuse angle side inner wall portion and the acute angle side inner wall portion are narrow, the curvature of the annular portion of the guide wire to be fed is small. Therefore, the guide wire is applied with strong ironing by being sent in a state with a small curvature. As a result, the folded back part of the guide wire tends to be bent unnecessarily.

On the other hand, in the guide wire shaping tool of the present invention, by sliding the sliding portion, and increasing the interval between the obtuse angle side outlet opposing portion and the acute angle side outlet opposing portion that makes a part of the sliding portion and the interval between the obtuse angle side inner wall portion and the acute angle side outlet adjacent portion that makes a part of the sliding portion, it is possible to shape the guide wire in a sent state so as to draw a gentle curve, and as a result, it is possible to increase the curvature of the folded back part. This can relax the ironing applied to the guide wire, and it is possible to avoid a state in which the folded back part is bent so as to hang down the neck.

In the guide wire shaping tool of the present invention, since the sliding portion is slidable along the rail portion, these intervals can be freely changed. This can adjust the curvature of the folded back part, and it is possible to adjust the degree of bending or the degree of warping of the folded back part as per desire of the user. The term warping mentioned here refers to a state in which the folded back part of the guide wire is warped in an orientation opposite to the orientation of folding back.

In this manner, the guide wire shaping tool of the present invention can be more easily shaped into a desired shape.

A main body portion may be divided into a first main body portion and a second main body portion, the inlet passage portion and the shaping portion may be provided in a recessed manner in a first dividing surface, which is a dividing surface of the first main body portion, and each of the main body portions may be configured such that the first dividing surface and a second dividing surface, which is a dividing surface of the second main body portion, can freely overlap or separate.

That is, since the second dividing surface of the second main body portion can be freely overlapped or separated with respect to the first dividing surface of the first main body portion in which the inlet passage portion and the shaping portion are provided in a recessed manner, the guide wire can be accurately guided with the first dividing surface and the second dividing surface being in close contact so as to overlap each other when the guide wire is formed. When removing the guide wire after shaping from the guide wire shaping tool, when performing cleaning, maintenance, and the like on the inlet passage portion, the shaping portion, and the like, it is possible to easily perform the work by exposing the dividing surfaces so as to be separated from each other.

As described above, the present invention provides a guide wire shaping tool that can obtain uniform ease of shaping process of a guide wire and quality of shaping regardless of the direction of a core wire inside a guide wire to be inserted into the guide wire shaping tool. The guide wire shaping tool and the guide wire shaping method that can efficiently shaping a desired shape including folding back in a J shape are provided.

### BRIEF DESCRIPTION OF THE DRAWINGS

FIG. 1A shows, in the left view, a transverse cross section of a guide wire in which a plastic jacket covers around a core wire, and shows, in the right view, a longitudinal cross section of the guide wire; FIG. 1B shows, in the left view, a transverse cross section of a guide wire in which a coil is wound around the core wire, and shows, in the right view, a longitudinal cross section of the guide wire;

FIG. 2A shows, in the upper view, a transverse cross section of a guide wire in a case where the long direction of the core wire and the bending direction of the guide wire are the same, and shows, in the lower view, a longitudinal cross section of the guide wire; FIG. 2B shows, in the upper view, a transverse cross section of a guide wire in a case where the short direction of the core wire and the bending direction of the guide wire are the same, and shows, in the lower view, a longitudinal cross section of the guide wire;

FIG. 3A shows a plan view of the guide wire shaping tool of the present invention; FIG. 3B shows a front view of the guide wire shaping tool;

FIG. 4 shows a guide wire shaped in a J shape;

FIG. 5A shows a left side view of the guide wire shaping tool of the present invention; FIG. 5B shows a right side view of the guide wire shaping tool;

FIG. 6 shows a plan view of the guide wire shaping tool of the present invention in a state where a sliding portion is removed;

FIG. 7A shows a back view of the sliding portion; FIG. 7B shows a plan view;

FIG. 8 shows a plan view of the guide wire shaping tool of the present invention in a state where the sliding portion is located on the backmost side;

FIG. 9A shows a plan view of the guide wire shaping tool of the present invention in a state where the sliding portion is slid slightly forward; FIG. 9B shows an enlarged plan view of the guide wire shaping tool;

FIG. 10A shows an enlarged plan view of the guide wire shaping tool of the present invention in which the sliding portion and the holding portion are omitted; FIG. 10B shows an enlarged plan view of the guide wire shaping tool of the present invention in a state where the tip end of the guide wire returns to an outlet portion;

FIG. 11A shows a plan view of the holding portion; FIG. 11B shows a left side view of the holding portion; FIG. 11C shows a front view of the holding portion;

FIG. 12 shows a plan view of in an example of the guide wire shaping tool of the present invention;

FIG. 13 shows a plan view of the guide wire shaping tool of the present invention in a state where the guide wire is caused to enter the inlet passage portion;

FIG. 14 shows a plan view of the guide wire shaping tool of the present invention in a state where the guide wire is further fed and the tip end advances the shaping portion rightward;

FIG. 15 shows a plan view of the guide wire shaping tool of the present invention in a state where the guide wire draws an annular shape;

FIG. 16 shows a plan view of the guide wire shaping tool of the present invention in a state where the guide wire is folded back;

FIG. 17 shows a plan view of the guide wire shaping tool of the present invention in a state of a process where the annular diameter of the guide wire staying inside the shaping portion is gradually reduced;

FIG. 18 shows an enlarged plan view of the guide wire shaping tool of FIG. 17;

FIG. 19 shows an enlarged plan view of FIG. 18 in which the holding portion and the guide wire are omitted;

FIG. 20 shows a plan view of the guide wire shaping tool of the present invention in a state where the sliding portion is further slid and the guide wire is further pulled out;

FIG. 21 shows an enlarged plan view of the guide wire shaping tool of FIG. 20;

FIG. 22A shows a state of further curving together with the folded back part of the guide wire; FIG. 22B shows a state in which the folded back part of the guide wire is warped;

FIG. 23 shows the guide wire shaping tool of the present invention in a state where shaping of the guide wire shaping tool is completed after the guide wire is pulled out;

FIG. 24 shows a flowchart of the guide wire shaping method of the present invention; and

FIG. 25 shows a left side view of the guide wire shaping tool of the present invention according to an embodiment in which the holding portion is fitted upward from the lower side.

### DETAILED DESCRIPTION OF PREFERRED EMBODIMENTS

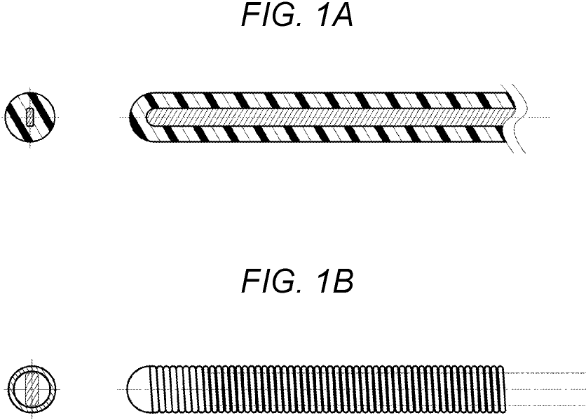

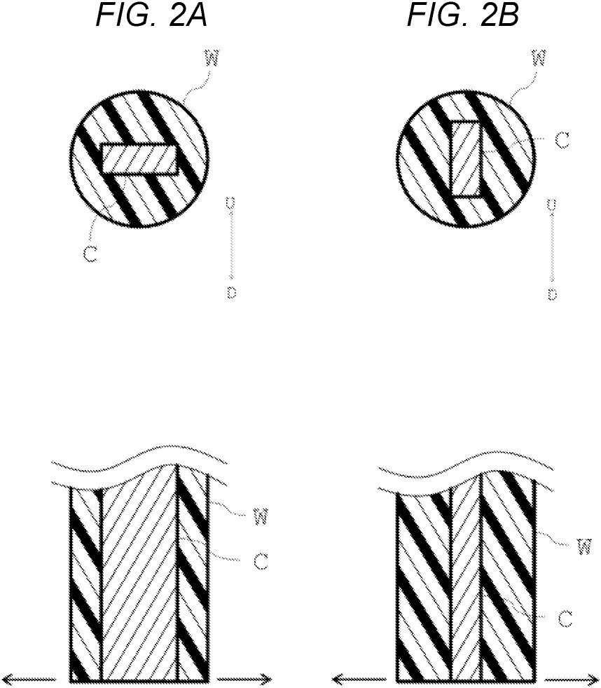

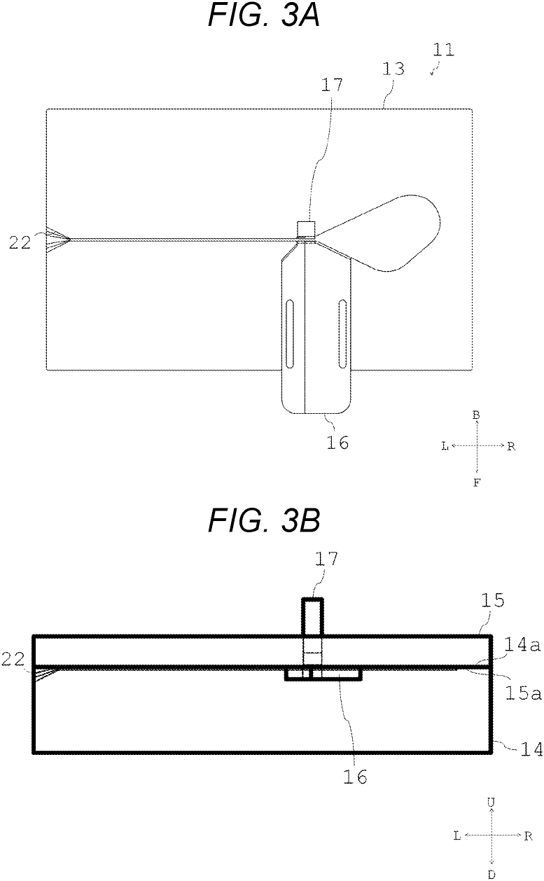

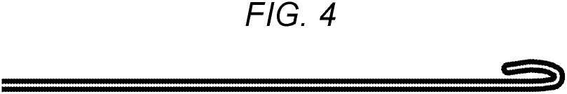

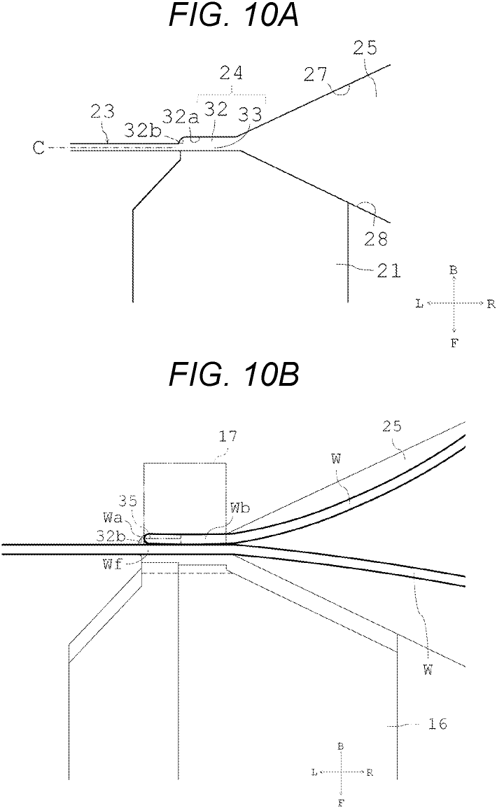

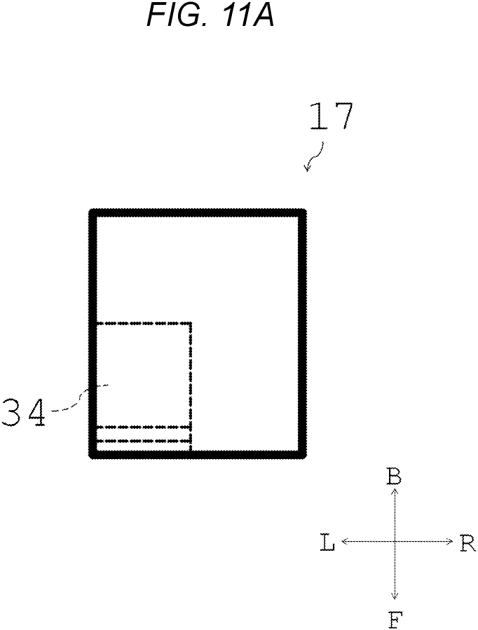

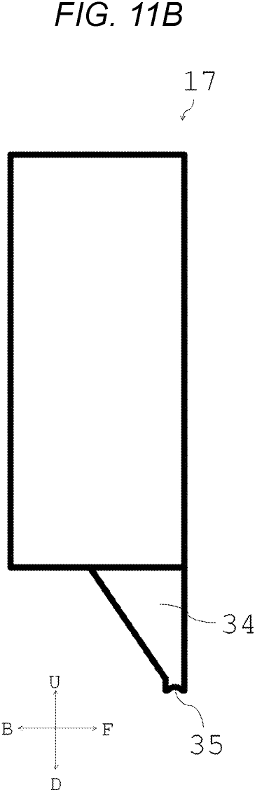

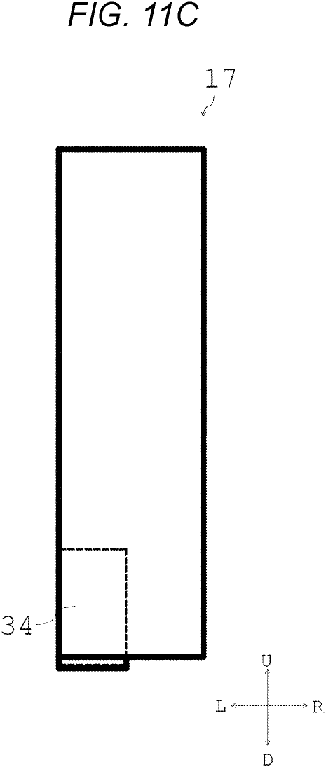

An embodiment of the present invention will be exemplified with reference to the drawings. In FIGS. 3, 11 denotes a guide wire shaping tool. The guide wire shaping tool 11 is used to shape the guide wire shown in FIGS. 1 and 2 into a so-called J shape as in the example shown in FIG. 4. The guide wire is indicated by reference sign W in FIG. 10B and subsequent drawings described below. In each drawing, arrow U represents upward U in the up-down direction of the guide wire shaping tool 11. Arrow D represents downward D in the up-down direction. The upward side is referred to as up side, and the downward side is referred to as down side. Arrow L represents leftward in the left-right direction. Arrow R represents rightward in the left-right direction. The leftward side is referred to as left side, and the rightward side is referred to as right side. Arrow F represents forward in the front-back direction. Arrow B represents backward in the front-back direction. The forward is referred to as front side, and backward is referred to as back side.

The guide wire shaping tool 11 has a main body 13 shown in the plan view of FIG. 3A. This main body 13 is divided in the up-down direction as shown in the front view of FIG. 3B and includes a first main body portion 14 arranged on the lower side and a second main body portion 15 arranged on the upper side. A first dividing surface 14a, which is a dividing surface of the first main body portion 14, and a second dividing surface 15a, which is a dividing surface of the second main body portion 15, are substantially horizontal surfaces. The first main body portion 14 is formed of a synthetic resin having characteristics such as impact resistance, abrasion resistance, heat resistance, and high dimensional accuracy, such as polyether ether ketone (PEEK). The second main body portion 15 is formed of a synthetic resin having high transparency such as an acrylic resin. Since the second main body portion 15 is transparent and see-through from above, the structure on the first dividing surface 14a is represented by a solid line in the plan view.

The first main body portion 14 and the second main body portion 15 are joined by a hinge or a bolt and a nut. Therefore, the first dividing surface 14a and the second dividing surface 15a can freely overlap or separate from each other by opening and closing the hinge or attaching and detaching the bolt and the nut.

The guide wire shaping tool 11 includes a sliding portion 16 and a holding portion 17. The sliding portion 16 is formed in a shape in which a substantially trapezoidal columnar solid that is flat in the up-down direction and a substantially rectangular parallelepiped solid that has a long direction in the front-back direction and is flat in the up-down direction are combined, and a part of the back side is fitted into a groove provided in a recessed manner in the first dividing surface 14a. The holding portion 17 is formed in a substantially quadrilateral columnar solid having a long direction in the up-down direction, and a part of the lower side is fitted into a hole 15b penetrating the second main body portion 15 in the up-down direction.

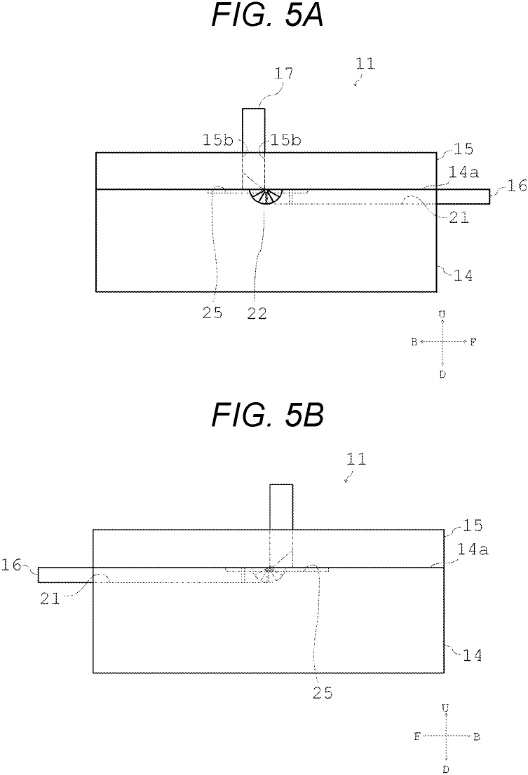

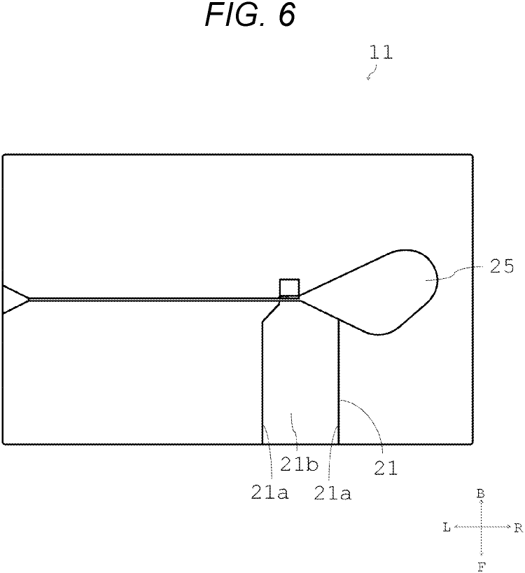

FIG. 5A shows a left side view of the guide wire shaping tool 11, and FIG. 5B shows its right side view. FIG. 6 shows a plan view in a state where the sliding portion 16 is removed. The first main body portion 14 includes a groove-shaped rail portion 21 provided in the front-back direction and into which a part of the back side of the sliding portion 16 is fitted. The rail portion 21 is provided in a recessed manner downward on the first dividing surface 14a as shown in FIGS. 5A and 5B, and has a leftward side wall 21a, a rightward side wall 21a, and an upward bottom wall 21b as shown in FIG. 6. These side walls 21a and the bottom wall 21b have a surface direction in the front-back direction. The rail portion 21 has a flat shape in the up-down direction, and the depth in the up-down direction is larger than the depth of a shaping portion 25.

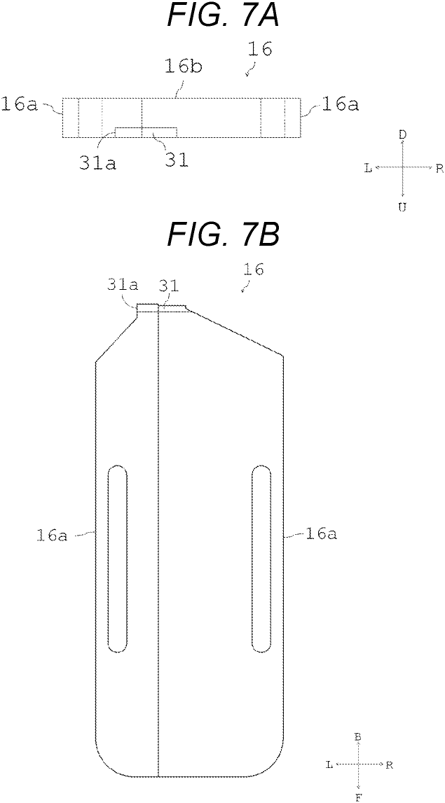

On the other hand, as shown in the back view of FIG. 7A and the plan view of FIG. 7B, the sliding portion 16 has a left side surface 16a and a right side surface 16a in the front-back direction and the up-down direction, and a bottom surface 16b in the front-back direction and the left-right direction. The sliding portion 16 has these left and right side surfaces 16a and 16a brought into contact with the left and right side walls 21a and 21a as well as the bottom surface 16b brought into contact with the bottom wall 21b, and can slide on the rail portion 21 to slide in the front-back direction. The up-down direction height of the sliding portion 16 is substantially the same as the up-down direction depth of the rail portion 21.

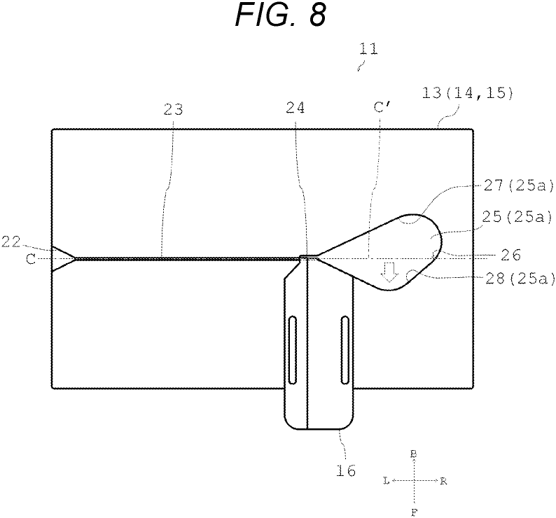

FIG. 8 is a plan view showing a state in which the sliding portion 16 is located on the backmost side and omitting display of the holding portion 17 for easy viewing. As shown in FIG. 5A, the first main body portion 14 has an opening portion 22 recessed rightward from the left side surface. The opening portion 22 forms a semi-conical space having a rightward apex. The tip end of a guide wire W is inserted into this opening portion 22. Then, an inlet passage portion 23, which is a substantially columnar space in the left-right direction as shown in FIG. 8, is formed so as to communicate with the opening portion 22. The inlet passage portion 23 is provided in a recessed manner downward in the first dividing surface 14a. Then, the inlet passage portion 23 has an outlet portion 24, which is a part located at the deepest side as viewed from the opening portion 22. That is, the outlet portion 24 is located on the rightmost side of the inlet passage portion 23.

Next, the first main body portion 14 has the shaping portion 25 communicating with the inlet passage portion 23 and expanding rightward from the outlet portion 24. Similar to the inlet passage portion 23, the shaping portion 25 is provided in a recessed manner downward in the first dividing surface 14a. The shaping portion 25 forms a flat space in the up-down direction on the side surface of FIGS. 5A and 5B as indicated by the hidden line. The shaping portion 25 has a substantially annular inner wall 25a forming its inner circumferential portion.

Here, an extension line C′ of a center line C of the inlet passage portion 23 intersects the inner wall 25a. The inner wall 25a of the part intersecting in this manner is called an extension line intersection portion 26. The extension line C′ is not orthogonal to the inner wall 25a but intersects at an obtuse angle at the extension line intersection portion 26. As shown in FIG. 8, the extension line C′ makes an obtuse angle with the inner wall 25a on the back side relative to the extension line intersection portion 26. At the same time, the extension line C′ makes an acute angle with the inner wall 25a on the front side relative to the extension line intersection portion 26. In the example of FIG. 8, the extension line C′ intersects the inner wall 25a at an angle of 129.0° at the extension line intersection portion 26. Of these inner walls 25a, the inner wall 25a that reaches the extension line intersection portion 26 through the back side from the outlet portion 24 and intersects the extension line C′ at an obtuse angle is called an obtuse angle side inner wall portion 27. The inner wall 25a that reaches the extension line intersection portion 26 through the front side from the outlet portion 24 and intersects the extension line C′ at an acute angle is called an acute angle side inner wall portion 28.

The acute angle side inner wall portion 28 has a shape bulging forward in an orientation away from the extension line C′ as indicated by a thick arrow in FIG. 8.

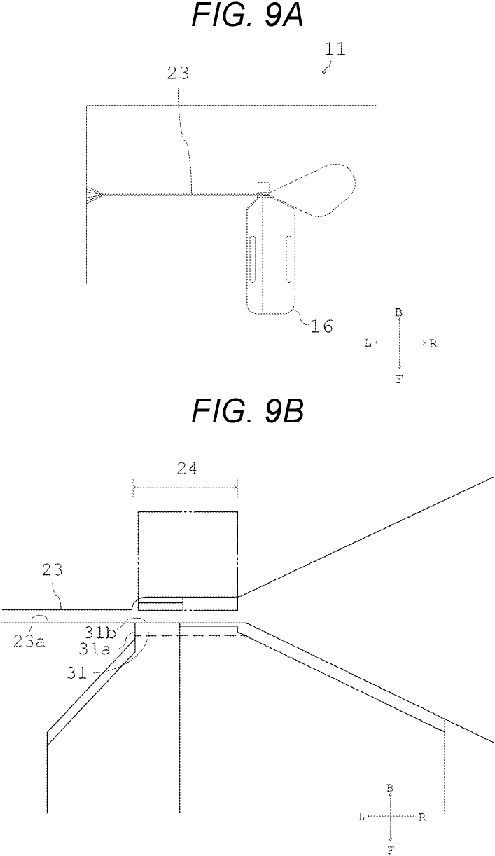

The sliding portion 16 has a back end portion 31 protruding backward as shown in FIG. 7B. The back end portion 31 is arranged at an upper part with respect to the thickness in the up-down direction of the sliding portion 16 as shown in FIG. 7A. The left side surface of the back end portion 31 is called a stopping surface 31a. Then, in a state where the sliding portion 16 is located on the backmost side as shown in FIG. 8, the back end portion 31 intersects with the center line C, and therefore the guide wire W entering rightward the inlet passage portion 23 through the opening portion 22 is blocked from further entering by the stopping surface 31a. On the other hand, FIG. 9A shows a state in which the sliding portion 16 is slid slightly forward. In this state, since a back surface 31b of the back end portion 31 is continuous left and right with a front end surface of the inner wall of the inlet passage portion 23 on the left side part relative to the outlet portion 24, or is located slightly on a front side relative to the inlet passage portion 23, the guide wire W is not blocked from entering by the stopping surface 31a. This state is called an open state of the inlet passage portion 23.

The back surface 31b of the back end portion 31 constitutes the acute angle side outlet opposing portion described below.

FIG. 9B is an enlarged plan view in an open state showing the holding portion 17 by an imaginary line. In this figure, the back surface 31b of the back end portion 31 is continuous left and right with a front end surface 23a of the inner wall of the inlet passage portion 23 on the left side part relative to the outlet portion 24.

FIG. 10A is an enlarged plan view near the outlet portion 24 in which the sliding portion 16 and the holding portion 17 are omitted. The outlet portion 24 has a width in the front-back direction that allows the guide wire W not shown in this figure to be doubly arranged front and back. This outlet portion 24 includes an obtuse angle side outlet portion 32, which is one side portion on a side having a substantially linear inner wall 32a continuous with the obtuse angle side inner wall portion 27, and an acute angle side outlet portion 33, which is one side portion on an opposite side of this obtuse angle side outlet portion 32. In this manner, the obtuse angle side outlet portion 32 and the acute angle side outlet portion 33 are arranged side by side with each other on the back side and the front side, respectively.

The obtuse angle side outlet portion 32 is closed at its left end by a closing portion 32b. On the other hand, the acute angle side outlet portion 33 is provided at a position extending rightward the inlet passage portion 23, and its left end communicates with the inlet passage portion 23.

FIG. 10B shows a state in which the guide wire W enters rightward the inlet passage portion 23, advances inside the shaping portion 25 while being bent counterclockwise, and then a tip end Wa returns to the outlet portion 24. At this time, since the acute angle side outlet 33 is occupied by the guide wire W on the front side, the tip end Wa is stopped rightward by the closing portion 32b after entering the obtuse angle side outlet portion 32. In this manner, since the outlet portion 24 has the obtuse angle side outlet portion 32 on the upper side and the acute angle side outlet portion 33 on the lower side, the guide wire W can be doubly arranged front and back. Of the two front and back side guide wires W in FIG. 10B, the guide wire W having returned to the outlet portion 24 on the rear side is called a back side guide wire Wb and is arranged inside the obtuse angle side outlet portion 32. The tip end Wa makes a part of the back side guide wire Wb. The other guide wire W on the front side is called a front side guide wire Wf, and the guide wire Wf passes through the acute angle side outlet portion 33 left and right.

FIGS. 11A, 11B, and 11C are a plan view, a left side view, and a front view of the holding portion 17, respectively. A part of the lower side of the holding portion 17 is fitted into the hole 15b of the second main body portion 15. The holding portion 17 has a lower end portion 34 protruding downward. The lower end portion 34 has a width of substantially half in the left-right direction of the holding portion 17 in plan view and is arranged to the left, and has a depth of substantially half in the front-back direction of the holding portion 17 and is arranged to the front. The lower end portion 34 is formed with the back surface being inclined such that the depth becomes smaller toward the lower side.

The lower end portion 34 has a concave portion 35 formed in a partial cylindrical shape in the left-right direction on the bottom surface. By the holding portion 17 indicated by an imaginary line in the plan view of FIG. 10B descends along the hole 15b, the concave portion 35 can press the tip end Wa and the guide wire W near the tip end Wa downward from the upper side. At this time, since the tip end Wa and the guide wire W near the tip end Wa are supported downward by the first main body portion 14 corresponding to the bottom wall of the obtuse angle side outlet portion 32, the tip end Wa and the guide wire W near the tip end Wa are pressed against and held by the main body 13 by the lower end portion 34.

However, the concave portion 35 is so thin in the left-right direction that the holding portion 17 can press downward only the back side guide wire Wb arranged inside the obtuse angle side outlet portion 32, and does not press the front side guide wire Wf. Therefore, meanwhile the back side guide Wb remains at the obtuse angle side outlet portion 32, the front side guide wire Wf can freely move in the left-right direction in the acute angle side outlet portion 33.

FIG. 12 shows an example of the dimension in a plan view of an example of the guide wire shaping tool 11. Here, the extension line C′ and the inner wall 25a intersect at an angle of 129.0°. However, the extension line C′ may intersect the inner wall 25a at an angle other than 129.0°, and may intersect the inner wall 25a at an angle of about 129.0° as long as the tip end Wa slides along the obtuse angle side inner wall portion 26 without being caught as the guide wire W is fed.

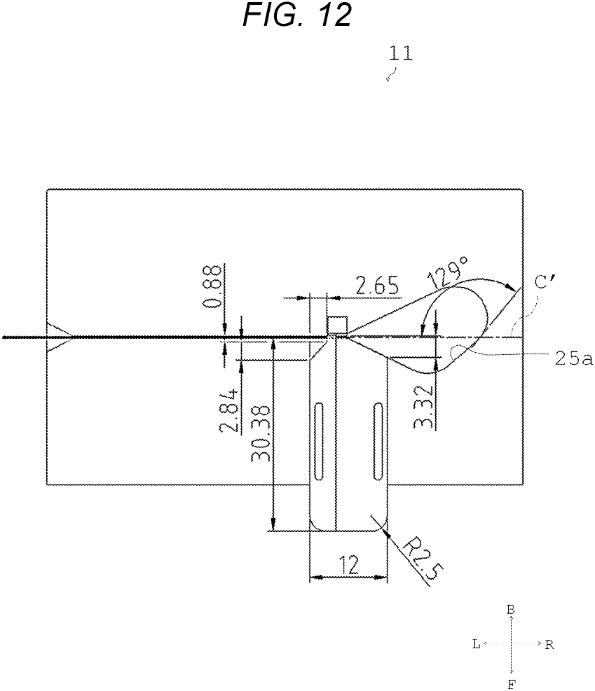

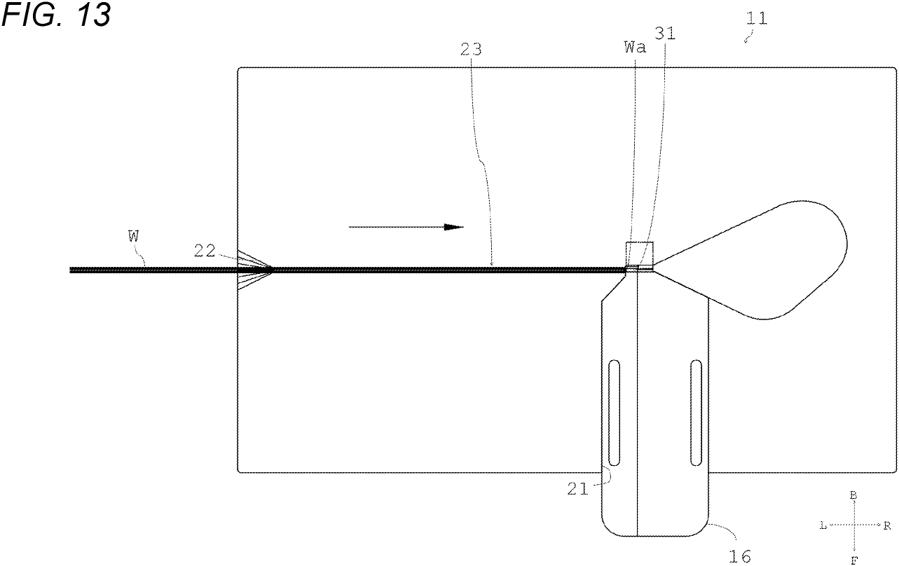

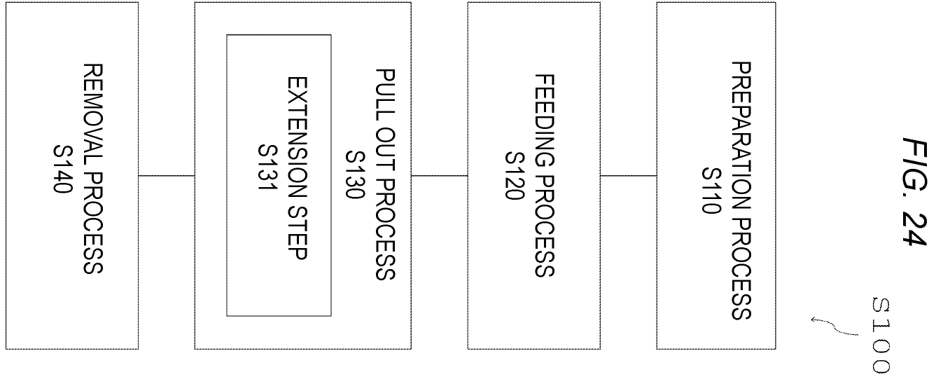

Hereinafter, a guide wire shaping method S100 in which the user folds back the tip end Wa of the guide wire W using the guide wire shaping tool 11 of the present invention and shapes the tip end part into a J shape will be described using plan views and enlarged plan views of FIGS. 13 to 21 and 23 in order. In these figures, the holding portion 17 is represented by an imaginary line, and a structure hidden by the holding portion 17 when originally viewed from above is also represented by a solid line. The guide wire shaping method S100 is also represented by the flowchart of FIG. 24.

FIG. 13 shows a state in which the first main body portion 14 and the second main body portion 15 are joined in the up-down direction. The process of preparing the guide wire shaping tool 11 and joining these main body portions 14 and 15 is called a preparation process S110.

Here, since the second dividing surface 15a of the second main body portion 15 can freely overlap or separate from the first dividing surface 14a of the first main body portion 14 in which the inlet passage portion 23 and the shaping portion 25 are provided in a recessed manner, the guide wire W can be accurately guided with the first dividing surface 14a and the second dividing surface 15a being in close contact with each other so as to overlap each other when the guide wire W is shaped.

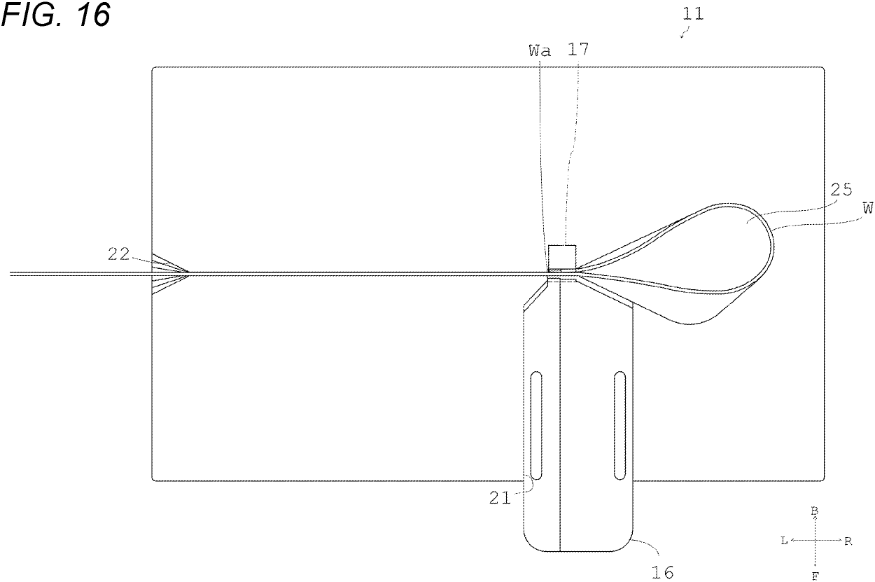

FIG. 13 shows a state in which the user inserts the tip end Wa into the opening portion 22, and is about to feed the guide wire W from the outside of the guide wire shaping tool 11 by causing the guide wire W to enter rightward the inlet passage portion 23. An entry direction of the guide wire W is indicated by an arrow. In this figure, the sliding portion 16 has moved to the backmost position on the rail portion 21 similarly to FIG. 8. Therefore, the tip end Wa cannot advance rightward beyond the stopping surface 31a of the sliding portion 16. The process from when the guide wire W is inserted into the opening portion 22 to when the tip end Wa abuts against the closing portion 32b and is stopped as shown in FIG. 16 described later is called a feeding process S120. In this feeding process S120, the holding portion 17 is raised, and the lower end portion 34 is not in contact with the guide wire W.

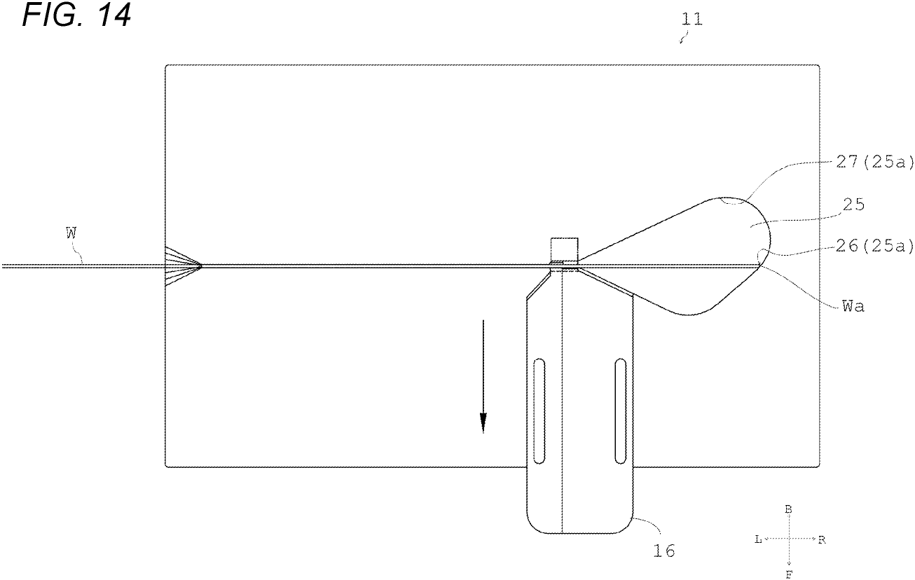

FIG. 14 shows a state in which the user slides the sliding portion 16 slightly forward and places the inlet passage portion 23 in the open state as shown in FIGS. 9A and 9B. In this state, the guide wire W can be further fed rightward, and the tip end Wa advances rightward in the shaping portion 25 through the acute angle side outlet portion 33. The rising holding portion 17 does not obstruct the travel of the guide wire W. Then, the tip end Wa travels substantially along the extension line C′ and abuts on the vicinity of the extension line intersection portion 26. The angle formed between the guide wire W and the inner wall 25a when the tip end Wa abuts on the vicinity of the extension line intersection portion 26 is substantially the same as the angle between the extension line C′ and the inner wall 25a. In this example, the angle formed with the obtuse angle side inner wall portion 27 is 129.0°, which is an obtuse angle. The present inventor has found that the guide wire W naturally advances while being bent in a side forming an obtuse angle with the traveling direction at the time of abutment. Due to this, the tip end Wa abuts on the vicinity of the extension line intersection portion 26, and then is bent counterclockwise in the figure and slides along the obtuse angle side inner wall portion 27. Then, the guide wire W in the shaping portion 25 is bent such that the entire guide wire W is annularly arranged.

Although FIG. 13 shows an example of a state in which the sliding portion 16 is first arranged at the backmost position, the sliding portion 16 may be arranged slightly on the front side from the beginning and the inlet passage portion 23 may be in the open state. In this case, the user can advance the tip end Wa into the shaping portion 25 from the beginning without sliding the sliding portion 16.

Here, as a result of intensive studies, the present inventor has found that when the tip end Wa of the inserted guide wire W abuts on the vicinity of the extension line intersection portion 26 and slides along the obtuse angle side inner wall portion 27 while being bent on the obtuse angle side, even if the long direction of the transverse cross section of the flat-plate-like core wire C inside the guide wire W is substantially the same as the front-back direction and the core wire C is located at a position as in FIG. 2A, the guide wire W rotates by 90° about its center axis, and has completed the present invention. As a result of the rotation, the long direction and the up-down direction of the cross section of the core wire C become substantially the same, and therefore the guide wire W is easily bent. It has been found that even when the long direction of the cross section of the core wire C and the bending direction of the guide wire W are not the same and form an angle of equal to or less than 90°, the guide wire W rotates by the angle of equal to or less than 90°. As a result of the rotation, similarly, the long direction and the up-down direction of the cross section of the core wire C become substantially the same, and therefore the guide wire W is easily bent as compared with before the rotation.

In this manner, regardless of the direction of the core wire C inside the guide wire W when inserted into the guide wire shaping tool 11, the guide wire W abuts on the vicinity of the extension line intersection portion 26 and slides along the obtuse angle side inner wall portion 27, whereby the guide wire W can change its attitude so as to be easily bent. Therefore, the guide wire shaping tool 11 can achieve uniform ease of shaping process of the guide wire W and quality of shaping.

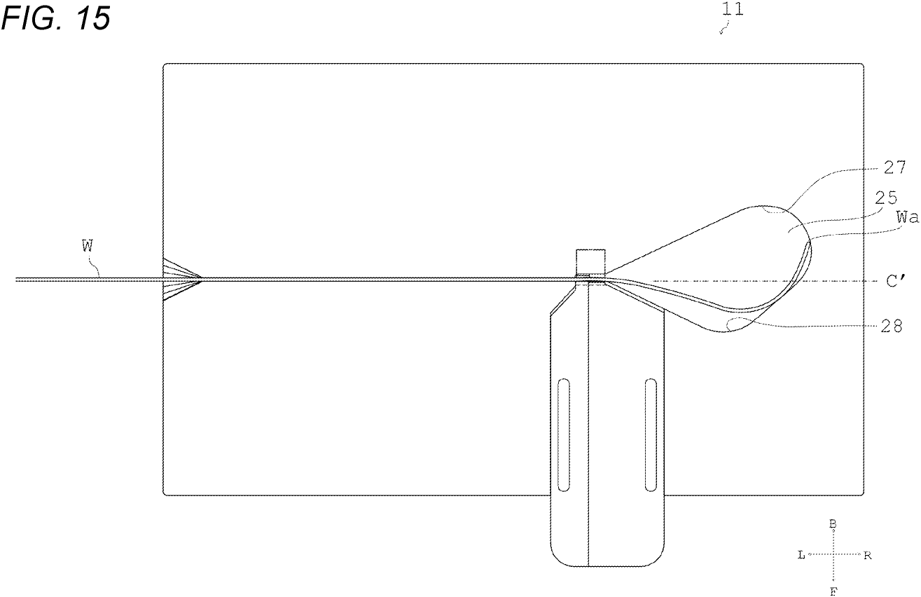

FIG. 15 shows a state in which the user continues to feed the guide wire W rightward, and the guide wire W in the shaping portion 25 draws an annular shape as a whole while the tip end Wa slides counterclockwise along the obtuse angle side inner wall portion 27. The acute angle side inner wall portion 28 located on the opposite side of the obtuse angle side inner wall portion 27 with respect to the extension line C′ has a shape bulging forward that is an orientation away from the extension line C′.

Therefore, the part continuous to the tip end Wa of the guide wire W can be curved so as to approach or contact the acute angle side inner wall portion 28. Due to this, as compared with the case where the part continuous to the tip end Wa is not curved, the tip end Wa can smoothly slide while making a shallower angle with respect to the wall surface direction of the obtuse angle side inner wall portion 27. Therefore, the guide wire W can more easily rotate about its central axis and can be more efficiently shaped. In this manner, the guide wire shaping tool 11 can achieve uniform ease of shaping process of the guide wire W and quality of shaping.

FIG. 16 shows a state in which the user continues to feed the guide wire W, the guide wire W draws a large annular shape in the shaping portion 25 to be bent counterclockwise, and the tip end Wa is folded back while changing the orientation from upward to leftward. As a result, the tip end Wa returns to the outlet portion 24. Then, similarly to FIG. 10B, the tip end Wa enters the obtuse angle side outlet portion 32 and then abuts against the closing portion 32b and is stopped.

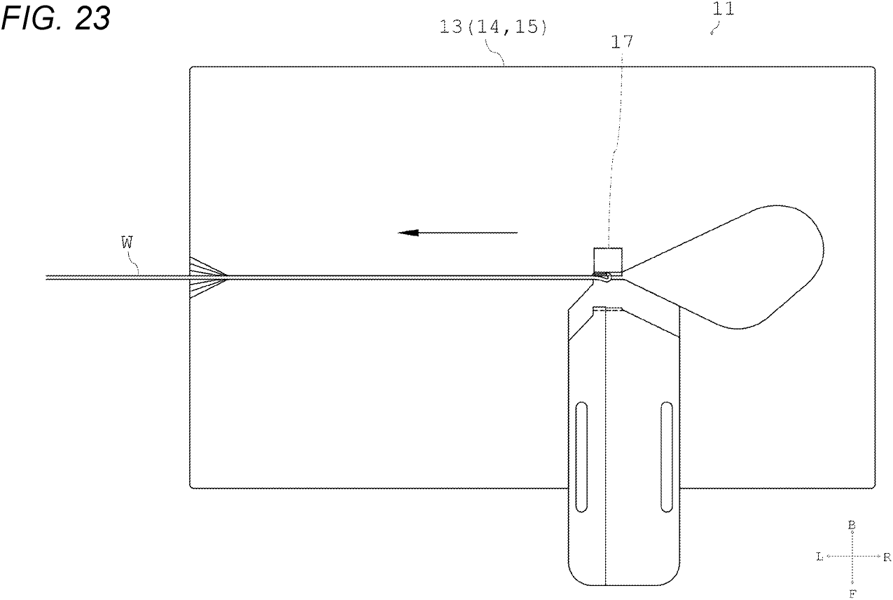

After the tip end Wa is stopped by the closing portion 32b in FIG. 16, the user lowers the holding portion 17 with the fingers and presses the concave portion 35 downward against the guide wire W to hold the guide wire W between the lower end portion 34 of the holding portion 17 and the first main body portion 14. The process from when the holding portion 17 is lowered in this manner to when the shaping of the guide wire W is completed as shown in FIG. 23 described later is called a pull out process S130.

In this pull out process S130, the back side guide wire Wb including the tip end Wa of the guide wire W and near it is pressed downward and held by the holding portion 17. In this state, the user pulls the guide wire W coming out of the opening portion 22 leftward, and pulls out the guide wire W from the inlet passage portion 23. Due to this, a part of the guide wire W annularly arranged inside the shaping portion 25 as shown in FIG. 16 is discharged from the shaping portion 25 through the acute angle side outlet portion 33. At this time, since the front side guide wire Wf is not pressed, the front side guide wire Wf can freely move leftward. The remaining portion of the guide wire W remaining inside the shaping portion 25 is shaped such that the annular diameter decreases as the annular surrounding decreases.

In this manner, the user of the guide wire shaping tool 11 first feeds the guide wire W, and its tip end Wa slides along the obtuse angle side inner wall portion 27 to reach the outlet portion 24, and then, conversely, partially pulls out the guide wire W from the opening portion 22. The guide wire W can be annularly shaped by reducing the annular diameter of the guide wire W inside the shaping portion 25. At this time, by the holding portion 17 holding the back side guide wire Wb, it is possible to reliably fix the position of the tip end Wa, and by the obtuse angle side outlet portion 32 of one side part holding the back side guide wire Wb, it is possible to pass, through the acute angle side outlet portion 33 of the other side part, the front side guide wire Wb on the side to be pulled out. Therefore, it becomes possible to perform shaping by reducing the annular diameter of the guide wire W more efficiently.

The user starts pulling out the guide wire W from the opening portion 22, and slides the guide wire W forward so as to press the sliding portion 16 forward with fingers to slide the rail portion 21.

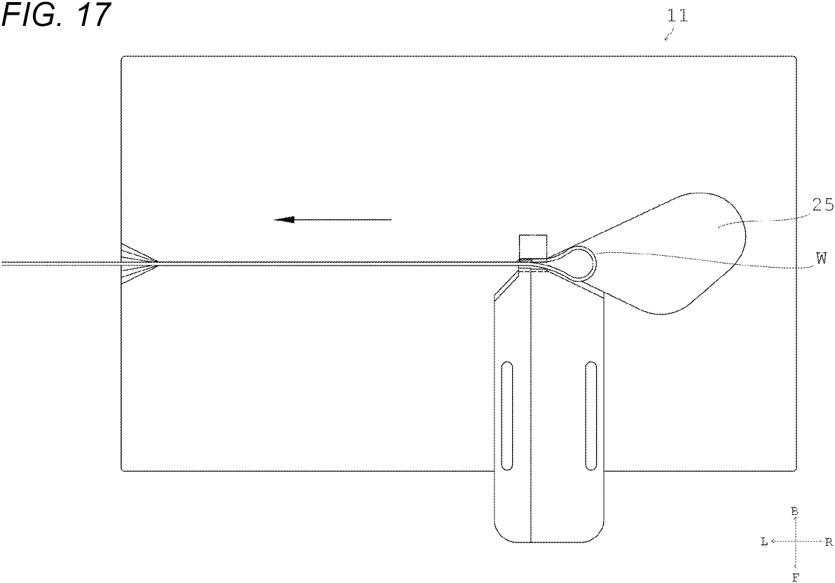

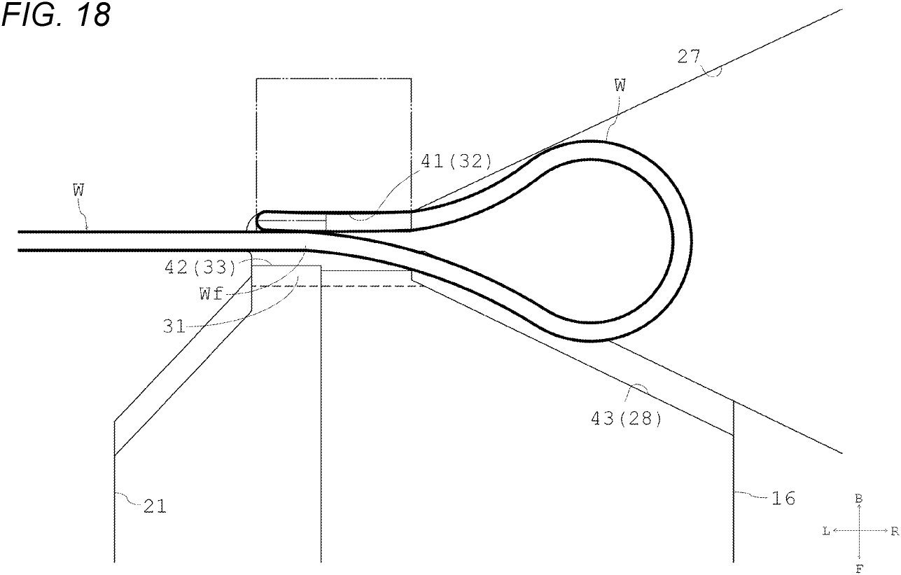

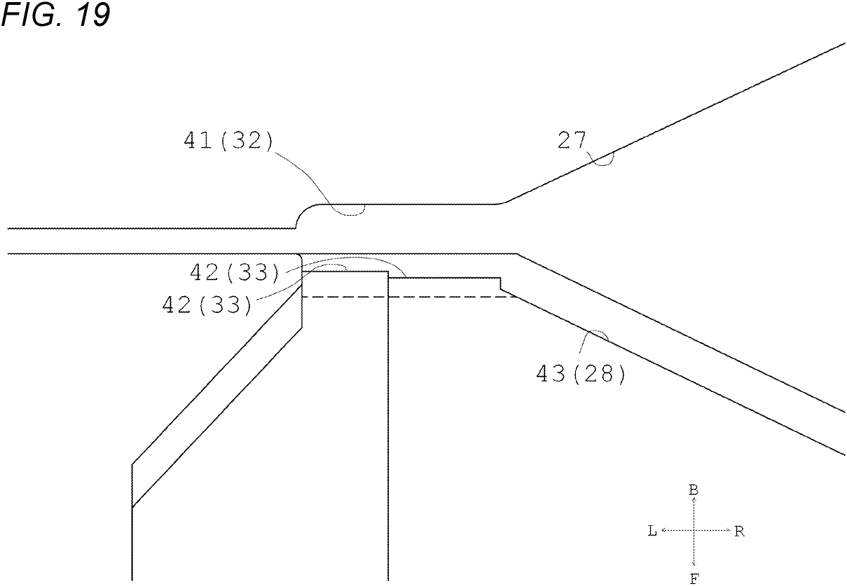

FIG. 17 shows a state in which the annular diameter of the remaining portion of the guide wire W remaining inside the shaping portion 25 is gradually reduced. FIG. 18 is an enlarged plan view in which the outlet portion 24 and a vicinity of the annular portion of the guide wire W in FIG. 17 is enlarged. An obtuse angle side outlet opposing portion 41, which is a forward inner wall of the obtuse angle side outlet 32, and an acute angle side outlet opposing portion 42, which is a backward inner wall of the acute angle side outlet 33, oppose each other in the front-back direction. A part of the acute angle side inner wall portion 28 adjacent to the acute angle side outlet opposing portion 42 is called an acute angle side outlet adjacent portion 43. The acute angle side outlet adjacent portion 43 forms a slope so as to be positioned on the front side toward the right side. The acute angle side outlet opposing portion 42 and the acute angle side outlet adjacent portion 43 constitute a part of the sliding portion 16. That is, the sliding portion 16 including the acute angle side outlet opposing portion 42 and the acute angle side outlet adjacent portion 43 forms a nest with respect to the first main body portion 14. FIG. 19 is a view in a state where the holding portion 17 and the guide wire W are not represented in order to make the obtuse angle side outlet opposing portion 41 and the acute angle side outlet opposing portion 42 easy to view.

When the sliding portion 16 slides forward as shown in FIGS. 17 and 18, the acute angle side outlet opposing portion 42 and the acute angle side outlet adjacent portion 43 move forward by a distance corresponding to sliding. Due to this, the interval between the obtuse angle side outlet opposing portion 41 and the acute angle side outlet opposing portion 42 expands in the front-back direction, and the interval between the obtuse angle side inner wall portion 27 and the acute angle side outlet adjacent portion 43 expands in the front-back direction. Therefore, the front side guide wire Wf is located on the front side as compared with the case where the acute angle side outlet opposing portion 42 and the acute angle side outlet adjacent portion 43 do not move forward. In this manner, the annular portion of the guide wire W can be expanded, and its curvature is greater than that in the case where the guide wire W has not moved.

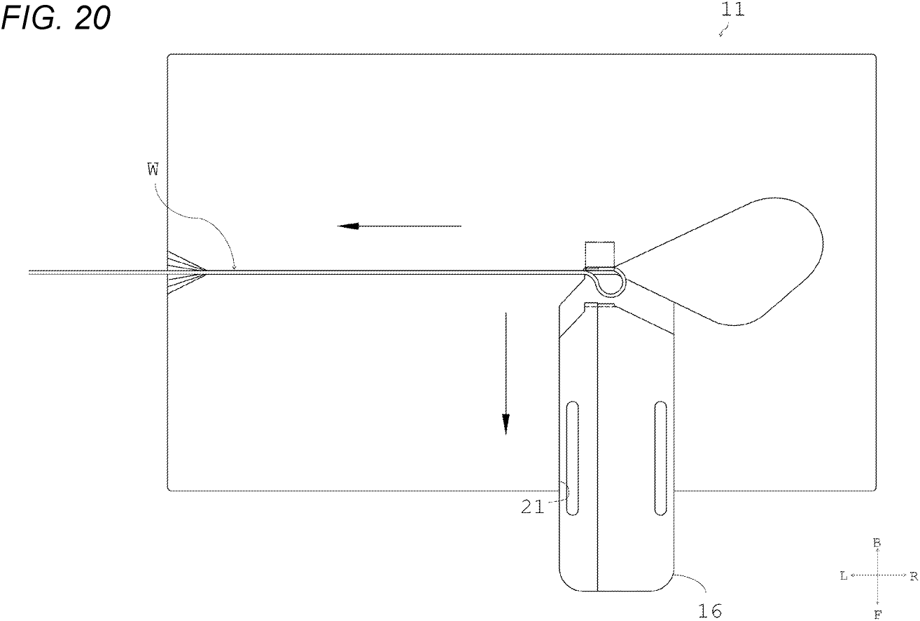

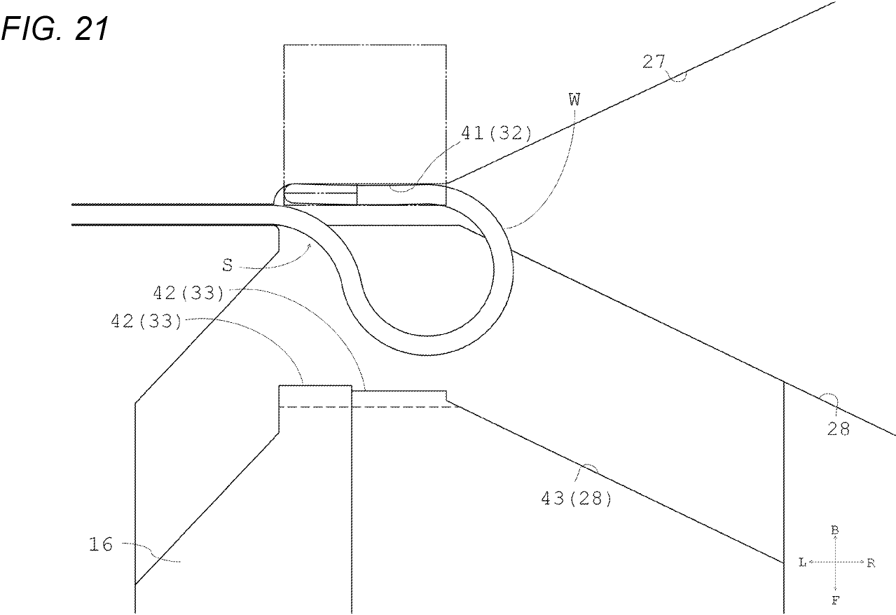

FIG. 20 shows a state in which the user further pulls out the guide wire W from the opening portion 22 than in the state of FIGS. 17 and 18, and further slides the guide wire W so as to press the sliding portion 16 forward to slide the rail portion 21. FIG. 21 is an enlarged plan view in which the outlet portion 24 and the vicinity of the annular portion of the guide wire W in FIG. 20 is enlarged similarly to FIG. 18. As described above, the step of expanding the interval between the obtuse angle side outlet opposing portion 41 and the acute angle side outlet opposing portion 42 and the interval between the obtuse angle side inner wall portion 27 and the acute angle side outlet adjacent portion 43 is called an extension step S131.

In this manner, by the sliding portion 16 further sliding forward, the longer the sliding distance is, the further the acute angle side outlet opposing portion 42 and the acute angle side outlet adjacent portion 43 move forward. Due to this, the interval between the obtuse angle side outlet opposing portion 41 and the acute angle side outlet opposing portion 42 further expands in the front-back direction, and the interval between the obtuse angle side inner wall portion 27 and the acute angle side outlet adjacent portion 43 further expands in the front-back direction. Therefore, the curvature of the annular portion of the guide wire W is much greater than that when the guide wire W has not moved.

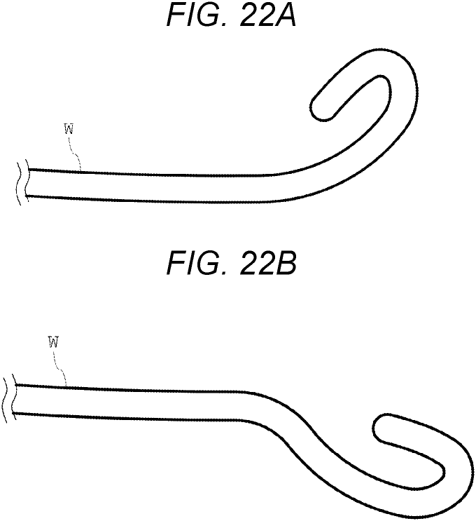

When shaping of folding back the guide wire W into the J shape is performed, if the force applied to bend the guide wire at the time of folding back is too large, stress in a specific orientation remaining after plastic deformation is large. Due to this, there is a case where after the guide wire W is removed from the guide wire shaping tool 11 in a removal process described later, the folded back part reaches a state of being bent to hang down the neck. That is, a state occurs in which the guide wire W in which the tip end Wa is folded back and shaped along the obtuse angle side inner wall portion 27 while being bent on the obtuse angle side is further curved in the orientation of folding back together with the folded back part. FIG. 22A shows an example of the state of further curving together with the folded back part of the guide wire in this manner. The state in which stress in a specific orientation due to such plastic deformation excessively remains is also referred to as state in which “ironing” is applied.

In particular, when shaping is performed with reducing the annular diameter of the guide wire W, if the interval between the obtuse angle side outlet opposing portion 41 and the acute angle side outlet opposing portion 42 and the interval between the obtuse angle side inner wall portion 27 and the acute angle side inner wall portion 28 are narrow, the curvature of the annular portion of the guide wire W to be fed is small. Therefore, the guide wire W is applied with strong ironing by being sent in a state with a small curvature. As a result, the folded back part of the guide wire tends to be bent unnecessarily upward in the figure as in FIG. 22A.

On the other hand, in the guide wire shaping tool 11 of the present invention, by sliding the sliding portion 16, and increasing the interval between the obtuse angle side outlet opposing portion 41 and the acute angle side outlet opposing portion 42 that makes a part of the sliding portion 16 and the interval between the obtuse angle side inner wall portion 27 and the acute angle side outlet adjacent portion 43 that makes a part of the sliding portion 16, it is possible to shape the guide wire W in a sent state so as to draw a gentle curve, and as a result, it is possible to increase the curvature of the folded back part. This can relax the ironing applied to the guide wire W, and it is possible to avoid a state in which the folded back part is bent so as to hang down the neck as in FIG. 22A.

In the guide wire shaping tool 11 of the present invention, since the sliding portion 16 is slidable along the rail portion 21, these intervals can be freely changed. This can adjust the curvature of the folded back part, and it is possible to appropriately adjust the degree of bending or the degree of warping of the folded back part as per desire of the user. The term warping mentioned here refers to a state in which the folded back part of the guide wire is warped downward in the figure as in the example shown in FIG. 22B. This warping is caused by ironing when the guide wire W is pulled and sucked into the inlet passage portion 23 from the shaping portion 25 and is bent outward in an annular shape conversely to the inside of the shaping portion 25. This point is exemplified by reference sign S in FIG. 21. As described above, the guide wire shaping tool 11 of the present invention can be more easily shaped into a desired shape.

FIG. 23 shows a state in which the guide wire W has been pulled out and its shaping has been completed. The process up to this state is the pull out process S130. After completion of shaping, the worker raises the holding portion 17 and stops holding of the guide wire W by pressing against the guide wire W. Then, the worker releases the joint between the first main body portion 14 and the second main body portion 15 by opening the hinge, removing the bolt and the nut, or the like. Due to this, by exposing the first dividing surface 14a, the worker removes, from the guide wire shaping tool 11, the guide wire W after the shaping. The process from raising the holding portion 17 to removing the guide wire W from the guide wire shaping tool 11 in this manner is called a removal process S140.

In this manner, in the guide wire shaping tool 11, the main body 13 is divided into the first main body portion 14 and the second main body portion 15, and these can be freely joined and the joining can be released, and therefore the user can easily remove the guide wire W after shaping from the guide wire shaping tool 11. Then, in order to shape another guide wire W next, the main body portions 14 and 15 can be joined again. Not only for removing the guide wire W but also when performing cleaning, maintenance, and the like on the inlet passage portion 23, the shaping portion 25, and the like, it is possible to easily perform work by exposing the dividing surfaces 14a and 15a so as to be separated from each other.

While the embodiment of the present invention has been exemplified above, the present invention is not limited to the embodiment, and various modifications can be made without departing from the gist of the present invention.

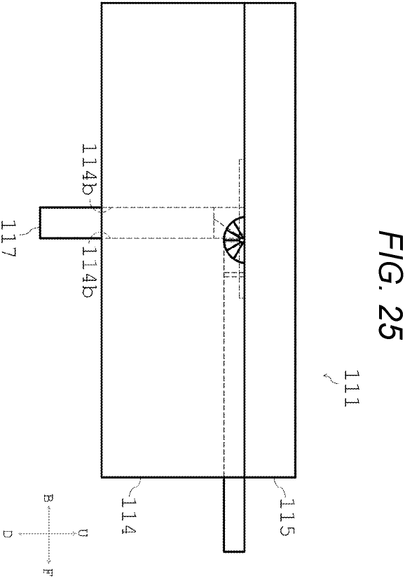

For example, in the above example, the embodiment has been described in which the holding portion 17 is fitted downward from the upper side of the guide wire shaping tool 11, but the holding portion may be fitted upward from the lower side of the guide wire shaping tool. Then, in the preparation process S110, a first main body portion 114, a second main body portion 115, and a holding portion 117 suitable for this may be prepared. FIG. 25 represents the left side surface of a guide wire shaping tool 111 in this different embodiment. In this guide wire shaping tool 111, a part of the upper side of the holding portion 117 is fitted into a hole 114b penetrating the first main body portion 114 in the up-down direction. This holding portion 117 can press the tip end Wa and the guide wire W near the tip end Wa upward from the lower side by rising along the hole 114b. At this time, since the tip end Wa and the guide wire W near the tip end Wa are supported upward by the second main body portion 115, the tip end Wa and the guide wire W near the tip end Wa are brought into a state of being pressed against the main body and held by the holding portion 117.

In the pull out process S130, even after passing through the state of FIG. 23, reciprocation by sliding of the sliding portion 16 may be repeated, or feeding and pulling out of the guide wire W may be repeated, and moreover these may be performed together. At this time, the back end portion 31 of the sliding portion 16 can press backward the folded back part of the guide wire W, and by slightly deforming this folded back part, the user can finely adjust the shape as desired.

The present invention can be used for a guide wire shaping tool and a guide wire shaping method for shaping a guide wire that is a flexible, wire-like instrument for facilitating insertion and indwelling of a catheter introducer into a blood vessel.

What is claimed:

1. A guide wire shaping tool comprising:
an inlet passage portion that is a substantially columnar space through which a tip end of a guide wire is inserted into an opening portion to allow the guide wire to pass;
a shaping portion that is a space communicating with the inlet passage portion through an outlet portion that is a portion located at a deepest part of the inlet passage portion and flatly expanding from the outlet portion;
an annular inner wall forming an inner circumferential portion of the shaping portion;
an extension line intersection portion that is a portion of the inner wall intersecting with an extension line of a center line of the inlet passage portion at an obtuse angle;
an obtuse angle side inner wall portion that is a portion of the inner wall extending from the outlet portion to the extension line intersection portion and located on a part angled at an obtuse angle with respect to the extension line; and
an acute angle side inner wall portion that is a portion of the inner wall extending from the outlet portion to the extension line intersection portion and located on a part angled at an acute angle with respect to the extension line,
wherein the guide wire is configured such that, as the guide wire is fed from the opening portion, the tip end that is sequentially inserted into the inlet passage portion and the shaping portion abuts against a vicinity of the extension line intersection portion, and at a time of this abutment, the guide wire slides along the obtuse angle side inner wall portion while being bent on the obtuse angle side, and the guide wire entirely is annularly arranged.

2. The guide wire shaping tool according to claim 1, wherein the acute angle side inner wall portion has a shape bulging in an orientation away from the extension line.

3. The guide wire shaping tool according to claim 2, further comprising:
an obtuse angle side outlet portion that is one side portion on a side having a substantially linear inner wall continuous with the obtuse angle side inner wall portion, of the outlet portion having a width that allows the guide wire to be doubly arranged;
an acute angle side outlet portion that is one side portion on a side opposite to the obtuse angle side outlet portion, of the outlet portion; and
a holding portion configured to press and hold the tip end and its vicinity against a main body of the guide wire shaping tool at the obtuse angle side outlet portion,
wherein the guide wire is configured to be partially pulled out from the opening portion, remaining in a state where the tip end and its vicinity are held by the holding portion, and a part of the guide wire annularly arranged inside the shaping portion is configured to be discharged through the acute angle side outlet portion and a remaining portion of the guide wire, other than the part of the guide wire, is configured to be annularly shaped with a reduced annular diameter.

4. The guide wire shaping tool according to claim 3, further comprising:
an obtuse angle side outlet opposing portion and an acute angle side outlet opposing portion that are inner walls opposing each other in the obtuse angle side outlet portion and the acute angle side outlet portion, respectively; and
a sliding portion in which a part including the acute angle side outlet opposing portion and an acute angle side outlet adjacent portion that is a part adjacent to the acute angle side outlet opposing portion of the acute angle side inner wall portion is nested, the sliding portion being slidable along a groove-shaped rail portion provided in a direction orthogonal to the center line,
wherein the sliding portion is configured to slide to increase an interval between the obtuse angle side outlet opposing portion and the acute angle side outlet opposing portion and an interval between the obtuse angle side inner wall portion and the acute angle side outlet adjacent portion.

5. The guide wire shaping tool according to claim 1, wherein the guide wire shaping tool has a main body portion which is divided into a first main body portion and a second main body portion, the inlet passage portion and the shaping portion are provided in a recessed manner in a first dividing surface, which is a dividing surface of the first main body portion, and each of the main body portions is configured such that the first dividing surface and a second dividing surface, which is a dividing surface of the second main body portion, can freely overlap or separate.

6. A guide wire shaping method comprising:
a preparation process of preparing a guide wire shaping tool including:
an inlet passage portion that is a substantially columnar space through which a tip end of a guide wire is inserted into an opening portion to allow the guide wire to pass,
a shaping portion that is a space communicating with the inlet passage portion through an outlet portion that is a portion located at a deepest part of the inlet passage portion and flatly expanding from the outlet portion,
an annular inner wall forming an inner circumferential portion of the shaping portion,
an extension line intersection portion that is a portion of the inner wall intersecting with an extension line of a center line of the inlet passage portion at an obtuse angle,
an obtuse angle side inner wall portion that is a portion of the inner wall extending from the outlet portion to the extension line intersection portion and located on a part angled at an obtuse angle with respect to the extension line, and
an acute angle side inner wall portion that is a portion of the inner wall extending from the outlet portion to the extension line intersection portion and located on a part angled at an acute angle with respect to the extension line; and
a feeding process of feeding the guide wire from the opening portion such that the guide wire has the tip end that is sequentially inserted into the inlet passage portion and the shaping portion abuts against a vicinity of the extension line intersection portion, and at a time of this abutment, the guide wire slides along the obtuse angle side inner wall portion while being bent on the obtuse angle side, and the guide wire entirely is annularly arranged.

7. The guide wire shaping method according to claim 6, wherein
in the preparation process, the guide wire shaping tool is prepared, wherein the guide wire shaping tool further includes:
an obtuse angle side outlet portion that is one side portion on a side having a substantially linear inner wall continuous with the obtuse angle side inner wall portion, of the outlet portion having a width that allows the guide wire to be doubly arranged,
an acute angle side outlet portion that is one side portion on a side opposite to the obtuse angle side outlet portion, of the outlet portion, and
a holding portion configured to press and hold the tip end and its vicinity against a main body of the guide wire shaping tool at the obtuse angle side outlet portion, and
the guide wire shaping method further comprises, after the feeding process, a pull-out process of holding the tip end and its vicinity by the holding portion and partially pulling out the guide wire from the opening portion, remaining in that state, discharging, through the acute angle side outlet portion, a part of the guide wire annularly arranged inside the shaping portion, and annularly shaping a remaining portion of the guide wire, other than the part of the guide wire, with a reduced annular diameter.

8. The guide wire shaping method according to claim 7, wherein
in the preparation process, the guide wire shaping tool is prepared, wherein the guide wire shaping tool further includes:
an obtuse angle side outlet opposing portion and an acute angle side outlet opposing portion that are inner walls opposing each other in the obtuse angle side outlet portion and the acute angle side outlet portion, respectively, and
a sliding portion in which a part including the acute angle side outlet opposing portion and an acute angle side outlet adjacent portion that is a part adjacent to the acute angle side outlet opposing portion of the acute angle side inner wall portion is nested, the sliding portion being slidable along a groove-shaped rail portion provided in a direction orthogonal to the center line, and
the pull-out process further includes an extension step of sliding the sliding portion, and increasing an interval between the obtuse angle side outlet opposing portion and the acute angle side outlet opposing portion and an interval between the obtuse angle side inner wall portion and the acute angle side outlet adjacent portion.

9. The guide wire shaping method according to claim 8, wherein
in the preparation process, the guide wire shaping tool is prepared, in which a main body portion is divided into a first main body portion and a second main body portion, the inlet passage portion and the shaping portion are provided in a recessed manner in a first dividing surface, which is a dividing surface of the first main body portion, and each of the main body portions is configured such that the first dividing surface and a second dividing surface, which is a dividing surface of the second main body portion, can freely overlap or separate, and the first main body portion and the second main body portion are joined such that the first dividing surface and the second dividing surface overlap each other, and
the guide wire shaping method further comprises, after the pull-out process, a removal process of removing the guide wire from the guide wire shaping tool after releasing joint between the first main body portion and the second main body portion such that the first dividing surface and the second dividing surface are separated from each other after the guide wire is shaped.
